(19) United States
(12) Patent Application Publication
(10) Pub. No.: US 2015/0306320 A1
(43) Pub. Date: Oct. 29, 2015

(54) METHOD AND DEVICE FOR DIRECTING BEEHIVE AIR, AND USE AS INHALER

(71) Applicant: Attila MARKUS, Isny-Neutrauchburg (DE)

(72) Inventor: Attila MARKUS, Isny-Neutrauchburg (DE)

(21) Appl. No.: 14/694,708

(22) Filed: Apr. 23, 2015

(30) Foreign Application Priority Data
Apr. 23, 2014 (DE) 102014005872.7

(51) Int. Cl. A61M 15/00; A01K 47/06

(52) U.S. Cl. CPC A61M 15/002; A01K 47/06

(57) ABSTRACT

A device and method for directing beehive air, in particular from at least one beehive, the beehive air is sucked up via at least one suction device, is conducted through at least one through flow device and is dispensed via at least one dispensing device. Further the beehive air is directed via at least one backflow check valve.

### CROSS-REFERENCE TO RELATED APPLICATIONS

This application claims priority to DE Patent Application No. 10 2014 005 872.7 filed Apr. 23, 2014, which is herein incorporated by reference.

### TECHNICAL FIELD

The invention relates to a method for directing beehive air (abbreviated to: bee air), in particular from at least one beehive.

### BACKGROUND

The inhaling of bee air for therapeutic purposes is known. However, the known methods and devices are either not flexible or the sucked-off air is not sufficiently prevented from flowing back into the beehive. During the inhaling of the beehive air, a backflow of the sucked-off air can occur especially when breathing out, the backflow disturbing the bees and making them aggressive.

### SUMMARY

The invention relates to a use of the device as a beehive air-conditioning unit or for improving the production of honey and/or as an inhaler device. It is an object of the present invention to provide a method, a device and use of a device, in which flexible directing of bee air is realized without reducing the quality of the bee air, and at the same time preventing the backflow of the sucked-off air. By preventing the backflow of air into the beehive, which may occur especially during the breathing-out phase of an inhalation, it is possible to prevent the bees from being disturbed in their habitat and thereby becoming aggressive during use of the device.

Advantageous developments of the invention are indicated in the dependent claims or are indicated below in conjunction with the description of the figures.

The invention includes the technical teaching that, in the case of a method for directing beehive air, in particular from at least one beehive, wherein the beehive air is sucked up via at least one suction device, is conducted through at least one through flow device and is dispensed via at least one dispensing device, the air flow direction in one direction, namely downwards from the beehive, is ensured. In the method, a fluidic connection to a beehive air reservoir is produced. The beehive air or, in more abbreviated form, bee air is stored, for example, in a beehive. In another embodiment, the bee air is stored in another vessel. The connection is realized, for example, via an attachment which is placed onto an opening in a beehive. After a fluidic connection to the bee air reservoir is realized, the bee air is sucked up via a suction device. The suction preferably takes place by means of an organism by breathing in, for example also via a fan, a pump or another fluid-conveying means. The sucked-up air is conducted through a through flow device. The through flow device has at least one backflow check valve or the like. The through flow device is connected fluidically here to the suction device, and the air flow direction is directed downwards from the beehive.

The bee air conducted through the through flow device is furthermore dispensed via a dispensing device. The dispensing is preferably carried out to an object connected to the dispensing device or to an organism, but can be carried out, for example, freely into an environment. For this purpose, the dispensing device has a connecting section. The dispensing device is connected to the object or organism, in particular connected releasably, via the connecting section. The directing, i.e. the suction, the conducting through and the dispensing, is controlled in the flow direction thereof. In one embodiment, the suction, the conducting through and/or the dispensing can be controlled in duplicate: the air flow rate and the air flow direction are controlled. In another embodiment, only the air flow direction is controlled. In the case of control of the air flow direction, control is preferably realized by at least one backflow check valve or the like. In the case of control of the air flow rate, the control is preferably carried out by activating the fluid-conveying device, i.e., the fan or the pump. In one embodiment, the control is carried out manually, semi-automatically and/or automatically. In another embodiment, the control is executed centrally. In one embodiment, the method can be carried out simultaneously with a plurality of bee air reservoirs and/or objects. For example, bee air is sucked up from a plurality of bee air reservoirs, either with one suction device or with a plurality of suction devices. The sucked-up bee air is conducted through one through flow device or a plurality of through flow devices. The bee air conducted there through is dispensed to one object or a plurality of objects via one dispensing device or a plurality of dispensing devices. The bee air reservoir is, for example, a beehive occupied by bees. In order not to adversely affect the bees, the control is carried out in a corresponding manner.

In one embodiment, it is provided that the beehive air is filtered via at least one filtering device. In particular during the dispensing to an object or organism, it is provided that only certain portions of the bee air are dispensed to the object. In particular, the filtering is undertaken in such a manner that, during the directing of the bee air, no bees pass to the object. From this purpose, in one embodiment, the bee air is filtered at the suction device, preferably at the starting section thereof and/or at the end section thereof. In another embodiment, it is provided that, in addition or alternatively, the filtering is carried out in or at the through flow device and/or in or at the dispensing device. The filtering provides coarse filtering and/or fine filtering. The coarse filtering is provided in particular in order not to allow bees to pass into the bee air flow. The fine filtering makes provision to direct undesired portions of the bee air. In particular, pollen is filtered with the fine filter. In particular, multi-stage filtering is provided. In one embodiment, the filtering is carried out as static filtering with an unchangeable filter. In another embodiment, the filtering is carried out as dynamic filtering, i.e. with a changeable filter.

The technical teaching of the invention furthermore makes provision for it to be provided that, in the case of a device for directing beehive air, in particular from at least one beehive, means for carrying out a method according to the invention are provided. The means are correspondingly suction devices, through flow devices, dispensing devices, filtering devices and devices preventing backflow.

In a further embodiment of the present invention, it is correspondingly provided that the means comprise at least one suction device with which the beehive air can be sucked up. In one embodiment, the suction device is provided as a separate device. In another embodiment, the suction device is designed as an integrated device, for example is designed as a device integrated into the through flow device and/or the dispensing device. In one embodiment, the suction device is of single-part design. In another embodiment, the suction device is of multi-part design. Furthermore, it is provided in one embodiment that the suction device is designed as a passive suction device, in which the bee air is directed by the breathing-in of the organism connected to the device and is directed in the same direction towards the person breathing in. In this form, a counter direction of the air flow, in the direction of the beehive, for example, due to breathing out, is prevented, for example, by a backflow check valve.

In another embodiment, the suction device is designed as an active suction device in which, for example, the bee air is sucked up by an actuator, such as a pump, a fan, a fluid-conveying unit or the like. In one embodiment, the suction device comprises one tube or pipe element or a plurality of tube or pipe elements through which the bee air can flow. In one embodiment, the tube element is designed as a flexible tube element. In another embodiment, the tube element is designed as a rigid tube element. With the tube element, the device, during interaction with a beehive, projects, for example, into the beehive. In a preferred embodiment, the suction device is designed as an attachment for a beehive. The attachment has the advantage of dispensing with projecting into the beehive, and therefore the bees in the beehive are not affected. In particular, the attachment is adapted to an opening of the beehive. The attachment here is preferably designed as an adapter plate or attachment plate which can be placed onto the beehive opening. The attachment plate is adapted to the beehive opening, and therefore said attachment plate at least partially projects into the beehive opening. The attachment plate laterally comprises at least one step with which the attachment plate rests on a region surrounding the beehive opening. In one embodiment, the step is designed as a frame which at least partially, preferably completely, surrounds the attachment plate laterally. The beehive opening is thus preferably closable by means of the attachment plate. The attachment plate here is connectable releasably to the beehive opening. The attachment plate is preferably formed from a natural material, in particular from wood. A passage opening through which the bee air can flow from the beehive or from an intermediate store to the through flow device is integrated in the attachment plate or the attachment.

In another preferred embodiment of the invention, it is provided that the means comprise at least one through flow device which is fluidically connected to the suction device and through which the sucked-up beehive air can be conducted. In one embodiment, the through flow device comprises a housing through which at least one through flow channel leads for the directing of the bee air. The through flow channel preferably contains the backflow check valve or the like. The through flow channel is preferably connected to the suck-off device. The through flow device is preferably connected separably to the suck-off device, in particular to the attachment or the attachment plate. The housing is preferably formed from a natural material, in particular from wood. In one embodiment, the through flow channel penetrating the housing opens into a connection for further components, in particular onto a connecting branch or the like. In another embodiment, the bee air is directed in the same direction in the through flow device.

In particular, at least one filtering device is arranged in the through flow device. For this purpose, the housing of the through flow device has a receptacle for the filtering device. The filtering device is arranged removably in the receptacle so that easy changing of the filter can be realized. The filtering device comprises at least one filtering material for filtering the bee air. Furthermore, the filtering device comprises a holder which holds the filtering material. The filtering material covers the fluidic connection between through flow device and suck-off device, and therefore all of the bee air flowing into the through flow device is directed through the filtering material and therefore the bee air is filtered.

In a further embodiment of the present invention, it is also provided that the means comprise at least one dispensing device which is fluidically connected to the through flow device and via which the beehive air conducted there through can be dispensed. In order to dispense the bee air in a targeted manner, a dispensing device is provided. In one embodiment, the dispensing device is provided as a separate device. In another embodiment, the dispensing device is designed as an integrated device, for example is designed as a device integrated in the through flow device and/or the suction device. In one embodiment, the dispensing device is of single-part design. In another embodiment, the dispensing device is of multi-part design. Furthermore, one embodiment makes provision for the dispensing device to be designed as an active dispensing device in which the bee air is actively dispensed via an actuator, such as a pump, a fan, a fluid-conveying unit or the like. In another embodiment, the dispensing device is designed as a passive dispensing device, in which, for example, the bee air is directed or sucked up by the organism connected to the device breathing. In order to facilitate the possibility of the bee air being sucked through the dispensing device by an organism and the air being breathed out into the environment, the dispensing device preferably contains a backflow check valve directed in manner corresponding to the task. In one embodiment, the air flow is preferably directed in the same direction in the dispensing device, for example by means of a plurality of backflow check valves. In one embodiment, the dispensing device comprises one tube element or pipe element or a plurality of tube elements or pipe elements through which the bee air can flow.

In one embodiment, the tube element is designed as a flexible tube element. In another embodiment, the tube element is designed as a rigid tube element. For connection to the through flow device and/or the suction device, the tube element has a corresponding connection section. The tube element is preferably placed with the connection section onto a connecting branch of the through flow device, and therefore a releasable connection is realized. For the targeted dispensing of sucked-up and conducted-through bee air to an object, for example an organism, such as a person, in particular for inhalation purposes, it is provided that the dispensing device, in particular the tube element, has a corresponding mouthpiece, nosepiece and/or a mask. A mask which, in one embodiment, covers the mouth and/or the nose, is preferably provided. The mask has a fluidic connection to the tube element. Furthermore, the mask has an outlet unit for letting out air which has already been inhaled, i.e. of used inhaled air. The outlet unit preferably functions at the same time as a backflow check valve. For the comfortable attaching of the mask to the person, the mask is preferably produced from a well-tolerated, flexible material. Furthermore, in one embodiment, the mask comprises an elastic band, one or more straps, and/or other holding elements, and therefore the mask does not have to be held manually by the person, but rather can be fastened, for example, to the person's head.

In yet another embodiment of the present invention, it is provided that the means comprise at least one control device with which the directing of the beehive air can be controlled, in particular can be controlled in an infinitely variable manner. In order to ensure that the harmful removal of bee air from the beehive is prevented, a control device which can reduce or shut off the air flow is provided. In particular, a stream of the bee air can be controlled via the control device.

In a further embodiment, a sensor unit is formed for detecting indicators with which a conclusion regarding the state of the bee air can be drawn. For example, in one embodiment, a sensor unit detects activity of the bees located in the beehive, for example the movement of said bees. A conclusion regarding the state of the bee air can be derived on the basis of a change in the movement or the activity of the bees, and therefore, if the need arises, a corresponding reduction in the sucked-off beehive air rate can be realized. In another embodiment, the sensor unit is designed as a temperature sensor, as a flow velocity sensor and/or as a pressure sensor. A state of the bee air, such as pressure, temperature, flow velocity and the like, can be detected via said sensor units. If one of the state variables reaches a critical range, the control device reacts and reduces the air flow. In another embodiment, the control device comprises a timer element with which the directing of the bee air can be carried out in a time-controlled manner. For example, the timer element is designed as a count-down timer which, for example after a certain period of time, for example after 30 minutes, switches off the directing of the bee air.

In a corresponding manner, the technical teaching of the present invention furthermore includes the fact that a use of a device according to the invention for directing beehive air, in particular from at least one beehive, as a beehive air-conditioning unit or for improving the production of honey and/or as an inhaler device in which beehive air is supplied in a controlled manner to an organism for inhalation, is provided.

The sucking off of the bee air firstly has the advantage and purpose that, in a beehive, a predetermined temperature and/or environmental condition prevails, which ensures, for example, optimum production of honey. Specifically at high temperatures, the temperatures prevailing in the interior of a beehive exceed 35° C. and even 40° C. In order to realize suitable air-conditioning in the interior of the beehive, increased activity of the bees is required, in particular by means of beating wings, in order, for example, to halt a temperature rise within a favourable range. By means of the suction device, for example, hot bee air is removed, and therefore an optimum bee air temperature is ensured in the interior. With the device, in addition to heat, moisture and further components are transported away from the beehive. Without the device, this operation has to be undertaken by the bees. By means of the use as an air-conditioning unit, an improvement in the production of honey is achieved. For example, at a quantity of approximately 3 kg of nectar, approximately 1 kg of honey is obtained. 2 kg of water and/or other components have to be removed or cannot be used for the honey. Without the device, the removal of the other components, including the water and the moisture, is carried out by the bees. With the device, this operation is essentially taken over by the device, and therefore the bees are not busy in the hive with air-conditioning tasks, but instead can collect further nectar. Accordingly, a greater quantity of honey can be produced since more nectar is collected. In addition, the removal of the other components, including water, is improved by the device, and therefore the quality of the honey is improved. A method for improving production of honey is therefore also realized.

Water vapour saturation in the beehive is approximately 100% during the blossom period. If the air in the beehive is sucked off by one or more organisms and, instead, colder air flows into the beehive from the outside with a substantially lower content of water, a considerable quantity of water can be removed from the honey and, as a result, the bees are assisted in the production of honey. A backflow check valve prevents the air which is breathed out and is identically warm and identically saturated with water vapour from passing back into the beehive. An example calculation of the quantity of water removed by the device depending on air humidity, degree of saturation, temperature and inhaled quantity of air shows how much water can be removed by the physiological respiration of connected people per day.

Water content at 20° C., degree of saturation 50%: 10 g/m³

Water content at 36° C., degree of saturation 100%: 50 g/m³

Difference in water content of the sucked-off air and air flowing in as replacement: 40 g/m³

Respiratory volume of an adult in 8 hours: 12 breaths/minute×0.5 l of air per breath×480 minutes=2880 litres

When 4 people are connected to a beehive (according to claim 8, FIG. 7), 2880 litres×4=11 520 litres of air are exchanged for dry and cool air by the use of the device.

In this case, 11 520 m³×40 g/m³=461 g of water are removed daily.

Exclusively by means of the use of the apparatus, 230 g of honey in a beehive therefore daily reach the desired content of water. A colony daily produces up to 1 kg of honey (or less), and the example calculation thus represents an increase in efficiency of the production of honey of 23%.

The method filed for patent protection dries and cools the beehive, and therefore the device with which the method is carried out can be used for improving the production of honey and for air-conditioning the beehive.

The device which is provided with a backflow check valve and is used in the above-described example calculation as an inhaler is independent of the power supply, is substantially simpler than a variant equipped with a fan, and the potential for technical malfunctions is substantially lower. The device with a backflow check valve, which device serves and is suitable especially for use by people, can demonstrate substantial advantages over “ApiAir” methods significant in the prior art.

The bee air is preferably not simply removed to the surroundings, but rather, on the contrary, is used for inhalation purposes. The positive effects of the bee air are used here. Diverse illnesses in people can be treated by the inhalation of bee air.

Therefore, both the production of honey can be improved and illnesses can be treated in users.

In particular, three filters are provided in the use as an inhaler. One filter is designed as a fine filter, in particular as a pollen filter. This prevents pollen from passing with the bee air to the user, for example when treating allergy sufferers. In addition, a coarse filter is provided. This prevents bees or coarse portions from passing into the device. In addition, a safety filter is provided. The latter is arranged, for example, in the tube element and/or upstream of the mask. This prevents bees which have inadvertently entered the device from passing to the user.

The device is preferably designed as a mobile device which carries out its tasks without an external supply of power. In one embodiment, the attachment is of changeable or exchangeable design such that said attachment can be placed onto a very wide variety of beehives.

The beehive air is directed by a tube to the user's inhalation mask. A backflow check valve or the like prevents the air which has been breathed out from flowing back and therefore disturbing the bees, or infection of the beehive by viruses, bacteria or other microorganisms. The user does not have any direct contact with the bees. The device can be used for treating respiratory disorders. In particular, the device can be used in order to alleviate and/or to cure allergies, croup, asthma and colds. The device can also be used to combat bronchitis, COPD, susceptibility to infection, immune deficiencies, chronic head pains and migraines.

Furthermore, measures improving the invention are indicated in the dependent claims or emerge from the description below of at least one exemplary embodiment of the invention, which exemplary embodiment is illustrated schematically in the figures. All of the features and/or advantages, including structural details, spatial arrangement and method steps, that emerge from the claims, the description or the drawing may be essential to the invention both by themselves and in a very wide variety of combinations.

### BRIEF DESCRIPTION OF THE DRAWINGS

FIG. 1 shows, in a schematic perspective view, a configuration of the device.

FIG. 2 shows schematically, in a cross-sectional view, a suction device.

FIG. 3 shows schematically, in a different perspective view, a through flow device.

FIG. 4 shows schematically, in a different perspective view, a backflow check valve of the device.

FIG. 5 shows, in a schematic perspective view, a configuration of the device where a plurality of suction devices 120 are used.

FIG. 6 shows, in a schematic perspective view, a configuration of the device where a plurality of through flow devices 140 are used.

FIG. 7 shows, in a schematic perspective view, a configuration of the device where a plurality of dispensing devices 160 are used.

### DETAILED DESCRIPTION

The device 100 comprises a suction device 120. The suction device 120 is connected fluidically to the interior of the beehive (not depicted here). For this purpose, the suction device 120 has a passage opening 121a (see 3). The passage opening 121a is formed in an attachment 122 of the suction device 120. The attachment 122 is designed as an attachment ring 122a. A frame serving as a step is formed at the edge of the attachment ring 122a in a manner surrounding the latter laterally, and a sealing ring 124 is incorporated medially, said sealing ring 124 fitting into the corresponding opening in the adapter plate 123 and preventing the beehive air from escaping. The attachment ring 122a rests by means of this frame on an edge of the adapter plate such that the latter is covered by the attachment 122. The adapter plate and the attachment plate 122a are arranged here offset in respect of the planes thereof with respect to each other such that, when the attachment 122 rests on the edge of the adapter plate, the attachment 122 projects in the direction of the beehive opening. The adapter plate (partially illustrated) for the attachment plate is of rectangular design, and therefore said adapter plate is adapted to the shape of the beehive opening, which is likewise rectangular. In a technical respect, the relationship between the beehive and the adapter plate is identical to that between the adapter plate and the attachment plate.

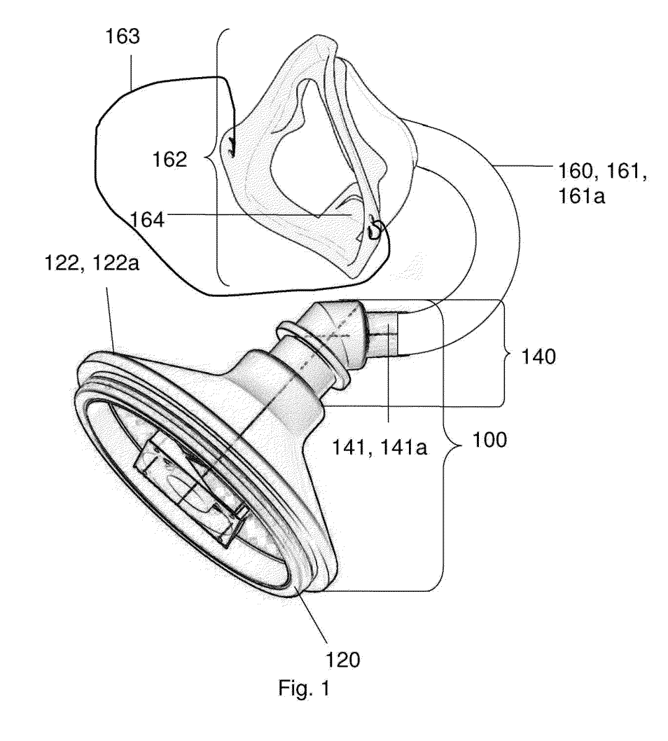

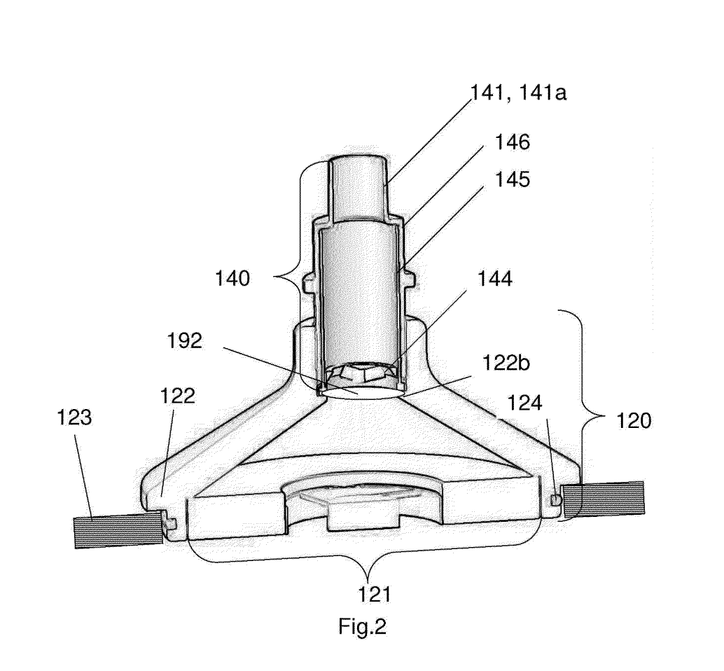

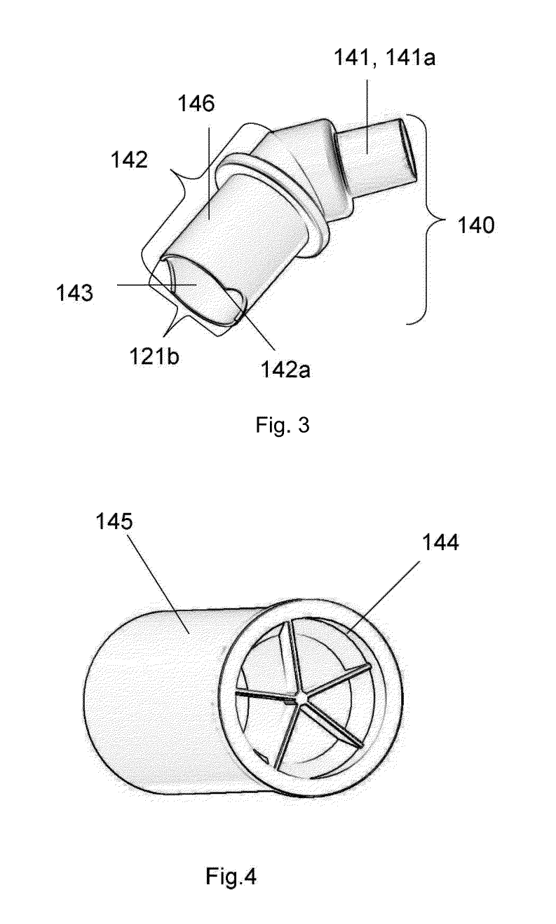

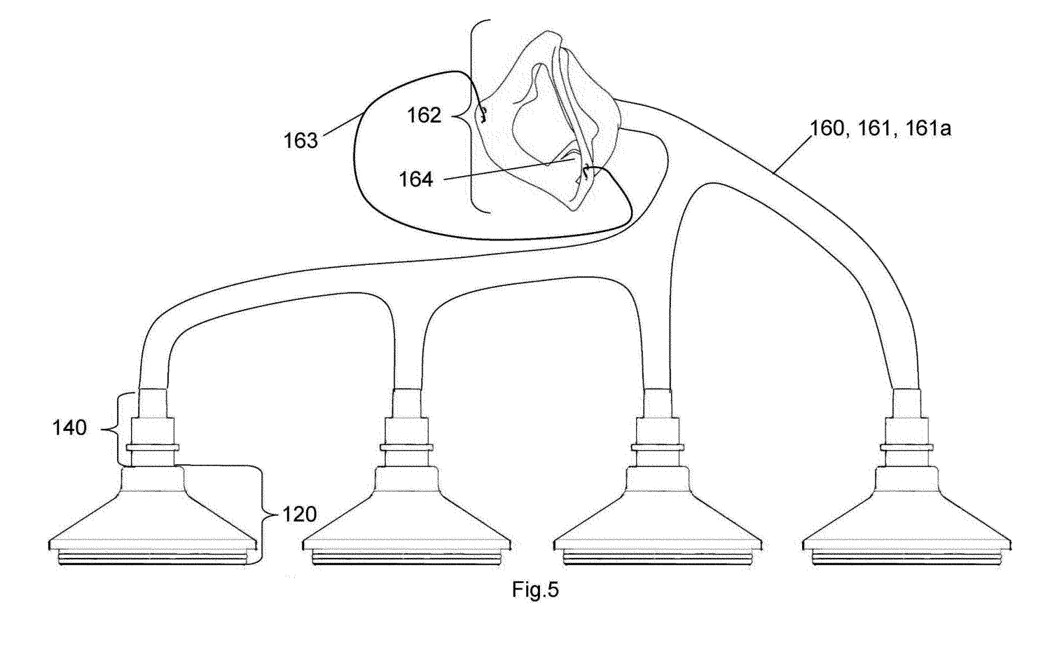

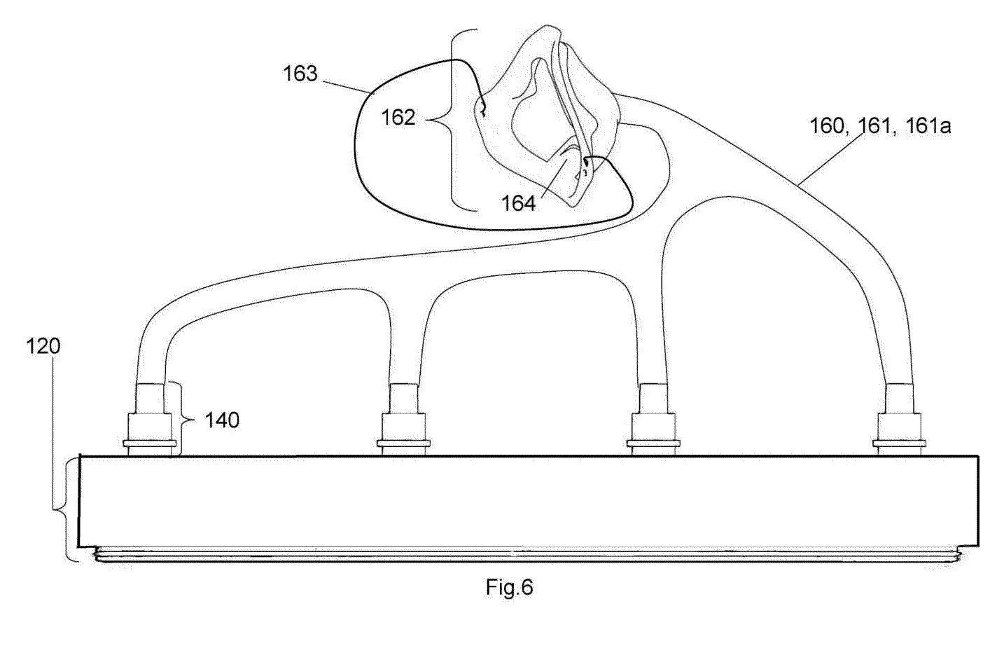

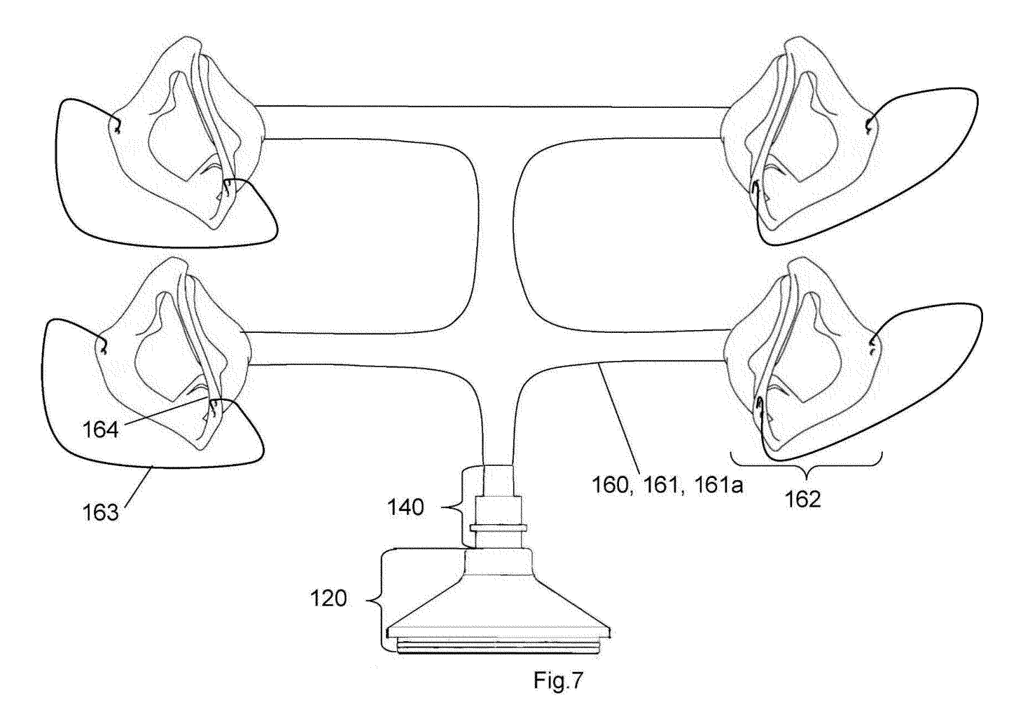

FIGS. 1 to 4 show, in various views and in various excerpts and detailed degrees, a device 100 for directing bee air and the components thereof. The device 100 comprises a suction device 120, a through flow device 140 and a dispensing device 160. According to one embodiment, the mixing of the bee air with ambient air during breathing in is prevented or inhibited by a backflow check valve 164

In the case of the configuration of the device that is illustrated in FIG. 1, the air is directed into a funnel-shaped suction device 120 and, at the neck of the funnel-shaped suction device 120, is directed further by a backflow check valve 144 placed into the through flow device 140. The air is directed further in a flexible tube element 161 and dispensed into the human body through a mask 162.

Furthermore, the device 100 comprises the through flow device 140. The through flow device 140 is fluidically connected to the suction device 120, and therefore bee air sucked up by the suction device 120 can be conducted through the through flow device 140. For this purpose, the through flow device 140 has a through flow channel 143. There is space for the backflow check valve 144 in the through flow channel 143. The cavity of the cylinder 145 mounted with respect to the backflow check valve 144 opens at one end into a connection 141 for the dispensing device 160, which connection is designed as a connecting branch 141a. The through flow device 140 has a housing 142. The housing 142 has a plurality of sections 142a-142c. The housing 142, in contact with one section 142a, rests on the attachment ring 122b after having been pushed into the cylindrical container 146 provided for it. The housing 142 here is connected to the attachment ring 122b in such a manner that the passage opening 122b is fluidically connected to an inlet of the through flow channel 143. Furthermore, the section 142a and the attachment ring 122b form a receptacle (not illustrated here) for the filtering material 192. The through flow channel extends over the three sections 142a-142c and opens into the connecting branch 141a. The connecting branch 141a is arranged on a side opposite the first section 142a.

In order now in a targeted manner to dispense the bee air which has been sucked up and conducted through, the dispensing device 160 is provided. The dispensing device 160 comprises a tube element 161 which is designed here as a flexible tube 161a. The tube element 161 has a suitable connection for the connecting branch 141a. In particular, the tube element 161 can be placed onto the connecting branch 141a. A mask 162 in the manner of a breathing mask or artificial respiration mask is arranged at the other end of the tube element 161. The mask contains an integrated backflow check valve 164. The mask 162 can be placed onto the user's nose and mouth and can be appropriately secured to the user's head via an adjustable and/or elastic rubber band 163 or the like, such that the mask 162 does not slip. The mask 162 has a fluidic connection to the tube element 161 such that the bee air can be supplied. In addition, the mask 162 has an outlet for letting out used inhaled air.

The device located between the suction device 120 and dispensing device 160 has to ensure that at least 50% of the sucked-up bee air arrives in the dispensing device 160, or the portion of undiluted bee air which arrives in the dispensing device 160 is at least 50%. The system is ideally airtight to the outside, that is to say, the suction device 120 receives 100% of the bee air and the dispensing device 160 dispenses 100% of the bee air.

The suction device 120 and the dispensing device 160 are connected rigidly to each other.

A backflow check valve has to prevent or inhibit air from flowing back into the beehive at an air pressure which is built up in the device by a person by breathing out.

The device has to be able to be adapted to a beehive or to a dispensing device connected to a beehive.

If the device is used for inhalation purposes, no part of the device that comes into contact with the bee air should dispense a substance, which is harmful to health, into the bee air, or a substance which is harmful to health should not reach a concentration which is harmful to health in the air dispensed by the dispensing device 160.

If the device is used for inhalation purposes, the dispensing device 160 or mask 162 has to ensure that the portion of undiluted bee air which enters the human body is at least 10%. The optimum value here is 100%.

Feature of the device indicated in one embodiment required for achieving the object according to the application, the device contains two or more suction devices 120.

Feature of the device indicated in one embodiment and required for achieving the object according to the application, the device contains two or more through flow devices 140.

Feature of the device indicated in another embodiment and required for achieving the object according to the application, the device contains two or more dispensing devices 160.

Feature of the device indicated yet another embodiment and required for achieving the object according to the application is provided with a dispensing device 160 or mask 162. The device contains a backflow check valve which either prevents or reduces the inflow of ambient air when breathing in or prevents or reduces the backflow of inhaled air in the direction of the suction device 120 when breathing out.

### LIST OF REFERENCE NUMBERS

100 Device
120 Suction device
121a Passage opening
121b Passage opening
122 Attachment
122a Attachment ring
122b Attachment ring
123 Adapter plate
124 Sealing ring
140 Through flow device
141 Connection
141a Connecting branch
142 Housing
142a (Housing) sections
143 Through flow channel
144 Backflow check valve
145 Cylinder
146 Cylindrical container
150 Fluid-conveying device
155 Power supply device
156 Plug
157 Plug connection
158 Fuse box
159 Power cable
160 Dispensing device
161 Tube element
161a Flexible tube
162 Mask
163 Rubber band
164 Backflow check valve of the breathing mask
192 Filtering material

What is claimed is:

1. Method for directing beehive air, in particular from at least one beehive, wherein the beehive air is sucked up via at least one suction device, is conducted through at least one through flow device and is dispensed via at least one dispensing device, wherein the air flow is directed via at least one backflow check valve.

2. The method according to claim 1, wherein the directing is facilitated by a fan or a pump.

3. The method according to claim 1, wherein the directing is controlled depending on a state of the beehive air.

4. The method according to claim 1, wherein the beehive air is filtered via at least one filtering device.

5. A device for directing beehive air, comprising:
at least one suction device at least one beehive,
at least one through flow device;
at least one dispensing device; and
at least one backflow check valve,
wherein the air flow is directed from the at least one suction device, through the at least one through flow device and out the at least one dispensing device, with the at least one backflow check valve preventing reverse flow.

6. The device according to claim 5, wherein the means comprise at least one suction device with which the beehive air can be sucked up.

7. The device according to claim 5, wherein the means comprise at least one through flow device which is fluidically connected to the suction device and through which the sucked-up beehive air can be conducted.

8. The device according to claim 5, wherein the means comprise at least one dispensing device which is fluidically connected to the through flow device and via which the beehive air conducted there through can be dispensed.

9. The device according to claim 5, wherein the dispensing device has a backflow check valve or the like and thus directs the air flow from the beehive further downwards.

10. A device for directing beehive air according to claim 5 providing a beehive air-conditioning unit for improving the production of honey.

11. A device for directing beehive air according to claim 5 providing an inhaler device in which beehive air is supplied in a controlled manner to an organism for inhalation.
